(19) United States
(12) Patent Application Publication
(10) Pub. No.: US 2006/0241605 A1
(43) Pub. Date: Oct. 26, 2006

(54) Intramedullary nail

(76) Inventors: Andre Schlienger, Arlesheim (CH); Markus Buttler, Oensingen (CH); Peter Senn, Waldenburg (CH)

Correspondence Address: JONES DAY, 222 EAST 41ST STREET, NEW YORK, NY, 10017-6702, US

(21) Appl. No.: 11/409,398

(22) Filed: Apr. 21, 2006

Related U.S. Application Data
(63) Continuation of application No. PCT/CH03/00683, filed on Oct. 21, 2003.

(51) Int. Cl. A61F 2/30

(52) U.S. Cl. 606/62

(57) ABSTRACT

This intramedullary nail (1), particularly intended for the tibia, has a proximal end portion (2), a distal end portion (3) intended for the introduction into the medullary canal and a central axis (6). The intramedullary nail (1) has an overall length L in the range of 200-500 mm and a curved section (4) with the length of G≦L. The curved section (4), with the length of G, has a curvature radius R in the range of 300-1300 mm and the L/R ratio is in the range of about 0.2 to about 0.8.

### RELATED APPLICATION DATA

The present application is a continuation of the U.S. National Stage designation of co-pending International Patent Application No. PCT/CH2003/000683, filed Oct. 21, 2003, the entire content of which is expressly incorporated herein by reference thereto.

### FIELD OF THE INVENTION

The invention relates to an intramedullary nail, and, more particularly, an intramedullary nail for use in the tibia.

### BACKGROUND OF THE INVENTION

CH-A 674 613 discloses a generic intramedullary nail having proximal and distal end portions, both angled away from a central part. The proximal end portion can have a curvature with a maximum radius of 220 mm.

By nature, the tibia is different for each patient such that the length of the tibia and the size of the tibial plateau, which are interdependent, are different for each patient. Consequently, a tibial intramedullary nail should also have parameters which change according to the length of the nail. Thus, a single curvature radius, used for every intramedullary nail regardless of length, is not optimal for insertion in the tibia because it will require an increased force and will lead to an increased loss of reduction.

### SUMMARY OF THE INVENTION

A preferred embodiment of the present invention seeks to remedy this problem. The object of the invention is to produce an intramedullary nail that takes the anatomical ratios of the tibia, relative to its length, into consideration and is optimal particularly regarding the course of the medullary canal.

This objective is achieved according to the invention with an intramedullary nail with an elongate nail body having a central longitudinal axis, a proximal end portion, a distal end portion configured and dimensioned for insertion into the medullary canal of the tibia, a total length L, where L is in the range of about 200 mm to about 500 mm, and a curved section having a length G, where G≦L. The curved section may have a radius of curvature R in the range of about 300 mm to about 1300 mm, and the ratio of L/R may be in the range of about 0.2 to about 0.8.

The advantages achieved by the invention are essentially that as a result of the intramedullary nail according to the invention

a) the insertion force is reduced for certain indications, especially in the case when the technique of closed drilling is used,

b) due to the low insertion force, a lesser loss of reduction is present,

c) after the insertion, the intramedullary nail is in the biomedically ideal position in the medullary canal,

d) as soon as the intramedullary nail comes against the posterior wall during the insertion, its curvature becomes effective (in the prior art, the intramedullary nail has to be either bent at this stage or the loss of reduction has to be taken into account).

In the case of one particular embodiment, the distal end of the intramedullary nail is constructed as a straight section having a length of l≦L. This brings about several advantages, namely:

a) a concurrence with the biomechanical axis,

b) the possibility of a distal fracture repair without loss of reduction, and

c) the displacement of distal bone fragments is avoided.

The length l of the distal end portion of the intramedullary nail is advisedly in the range of 0.20-0.55 L, preferably in the range of 0.25-0.50 L.

In the case of a particular embodiment, the curved section includes with the straight section an angle α, that is in the range of about 7° to about 12° and preferably in the range of about 8° to about 10°. In still another embodiment, curved section includes with the straight section an angle α that is in the range of about 8° to about 9°. Together with the distal end section and the special curvature radius this will result in an optimum position of the intramedullary nail in the medullary canal relative to the entry point of the intramedullary nail.

In the case of a particular embodiment, the cross-section, taken at a right angle to the central axis of the intramedullary nail, is not circular and has preferably an oval or elliptical construction. By virtue of this the intramedullary nail can be introduced deeper into the medullary canal until it reaches the posterior wall.

In the case of a further embodiment, the intramedullary nail has a longitudinal bore that is coaxial with the central axis. This makes the introduction of the intramedullary nail possible by a guide wire, that can also be used for the drilled out operating technique.

In the case of still another embodiment, the proximal end portion of the intramedullary nail is constructed as a straight section with a length of P≦L. This has advantages as far as production technology is concerned. The length P of the proximal end portion is advisedly in the range of ⅙ L to ⅓ L.

In the case of a further embodiment, in the region of the proximal end portion at least one locking hole, extending transversely to the central axis, is present.

In the case of another embodiment, in the region of the distal end portion at least one locking hole, extending transversely to the central axis, is present. By using a locking element, introduced through the locking hole, both a positional fixing in the axial direction, i.e. fixing against axial dislocation, and a rotational fixing, i.e. fixing against torsional dislocation, is affected.

In the case of a further embodiment, the curvature radius R of the curved section is in the range of 350-1200 mm, preferably in the region of 400-1100 mm. The L/R ratio may be in the range of 0.3 to 0.7, preferably in the range of 0.4 to 0.6.

In the case of another embodiment, two locking holes, extending transversely to the central axis, are present in the region of the distal end portion. This will bring about several advantages:

a) the possibility of locking in several directions,

b) an angularly stable fixing of the bone fragments relative to one another,

c) a more favourable absorption of the bending moments and axial and torsional loads.

In the case of a further embodiment, the two locking holes, extending transversely to the central axis, include between them an angle of about 45° to about 90°.

In the case of a further embodiment, the distal end portion has three locking holes, while the middle locking hole is at a different distance from the other two holes. The advantage of this arrangement is that none of the axes of the three locking holes can potentially become an axis of rotation. This results in reduced play between the intramedullary nail and the locking elements.

The implanting method is briefly described in the following based on a cannulated intramedullary nail:

step A: establishing and holding the optimal reduction, depending on the type of fracture,

step B: opening the medullary canal with the aid of an opening instrument, so that the entry angle and the orientation relative to the medullary canal, to suit the operating technique employed, agree,

step C: introducing a guide wire up to the distal, future end position of the intramedullary nail and determining the length of the required intramedullary nail,

step D: the intramedullary nail, pre-assembled on the insertion handle, is introduced into the medulla through the entry channel via the guide wire,

step E: after checking the axial position of the intramedullary nail and the reduction, the intramedullary nail is locked by using the locking options.

### BRIEF DESCRIPTION OF THE DRAWINGS

The invention and developments of the invention are explained in detail in the following based on the partly schematic illustrations of a preferred embodiment, in which:

FIG. 1 shows a longitudinal section through the intramedullary nail; and

FIG. 2 shows a cross-section taken along line II-II of FIG. 1.

### DETAILED DESCRIPTION OF THE PREFERRED EMBODIMENTS

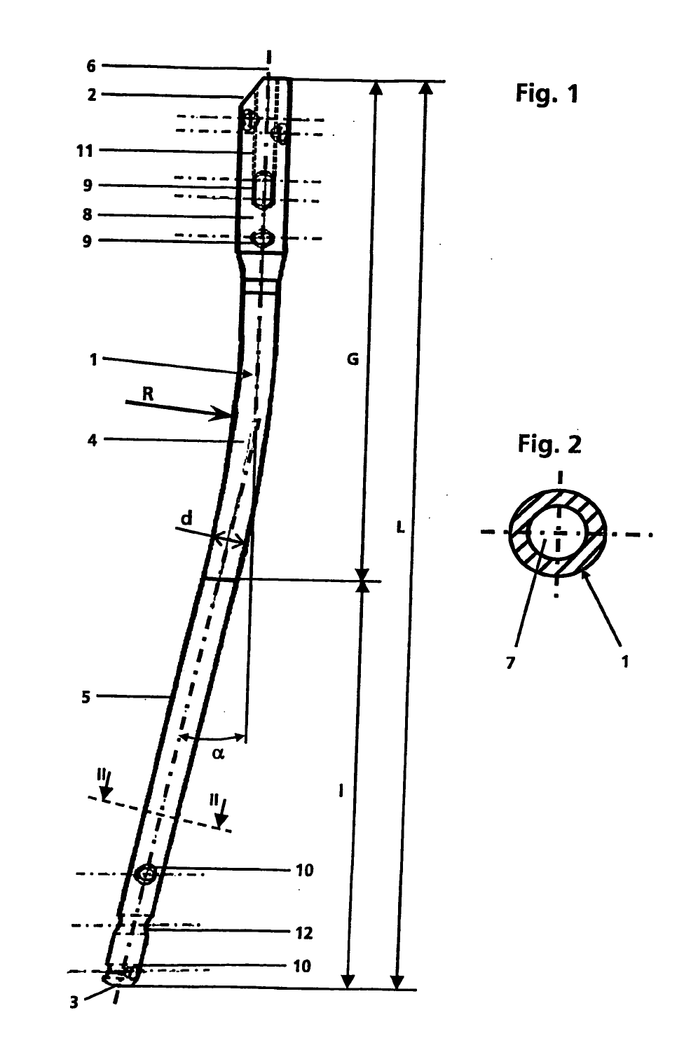

The intramedullary nail 1, illustrated in FIGS. 1 and 2, is intended to be used with the tibia. It has a proximal end portion 2, a distal end portion 3 intended for the introduction into the medullary canal, and a central axis 6. The proximal end portion 2 is provided with a threaded bore 11 to accept a conventional assistance. In one preferred embodiment, the overall length L of the intramedullary nail 1 is 255 mm. Furthermore, the intramedullary nail may have a curved section 4 with a length G=127.5 mm, and having a curvature radius R of 380 mm. Thus the L/R ratio is 0.67. After the intramedullary nail 1 has been implanted, the curvature of the intramedullary nail in the plane of the drawing corresponds to the anatomical medial-lateral plane, i.e. after the implantation the intramedullary nail 1 is bent in the anterior-posterior direction.

The distal end portion 3 is a straight section 5 with the length of l=127.5 mm. The curved section 4 includes an angle α=8° with the straight section 5. The intramedullary nail further has a longitudinal bore 7, that is coaxial with the central axis 6.

The proximal end portion 2 is constructed as a straight section 8 with a length of P=75 mm, such that P=0.3 L.

In the region of the proximal end portion 2 of the intramedullary nail 1 there are two locking holes 9 extending transversely to the central axis 6, with one of the holes constructed as a slot to enable compression of the fracture.

In the region of the distal end portion 3 there are three locking holes 10, 12, 10 extending transversely to the central axis 6, which are provided in different radial directions and include an angle of 90° with one another. At the same time the middle locking hole 12 is at a different distance to the other two locking holes 10, i.e., it is not equally-spaced between them.

While the present invention has been described with reference to the preferred embodiments, those skilled in the art will recognize that numerous variations and modifications may be made without departing from the scope of the present invention. Accordingly, it should be understood that the embodiments of the invention described above are not intended as limitations on the scope of the invention, which is defined only by the following claims.

What is claimed is:

1. An intramedullary nail for use in repairing bone fractures comprising:
an elongate nail body having
a central longitudinal axis,
a proximal end portion,
a distal end portion configured and dimensioned for insertion into the medullary canal of the tibia;
a total length L, where L is in the range of about 200 mm to about 500 mm; and
a curved section having a length G, where G≦L,
wherein the curved section has a radius of curvature R in the range of about 300 mm to about 1300 mm, the ratio of L/R is in the range of about 0.2 to about 0.8, and the distal end portion is a substantially straight section having a length l, where l≦L.

2. The intramedullary nail of claim 1, wherein the length l of the substantially straight distal end portion is in the range of about 0.20 L to about 0.55 L.

3. The intramedullary nail of claim 2, wherein the length l of the substantially straight distal end portion is in the range of about 0.25 L to about 0.50 L.

4. The intramedullary nail of claim 1, wherein the curved section is defined by first and second end points, and tangents taken at the first and second end points of the curved section include an angle α in the range of about 7° to about 12°.

5. The intramedullary nail of claim 4, wherein the tangents taken at the first and second end points of the curved section include an angle α in the range of about 8° to about 100.

6. The intramedullary nail of claim 1, wherein a cross-section taken perpendicular to the longitudinal central axis has a non-circular shape.

7. The intramedullary nail of claim 6, wherein the non-circular shaped cross-section is oval or elliptical.

8. The intramedullary nail of claim 1, wherein the nail body further comprises a longitudinal bore coaxial with the central axis.

9. The intramedullary nail of claim 1 wherein the proximal end portion is substantially straight and has a length P, where P≦L.

10. The intramedullary nail of claim 9, wherein P ranges between about ⅙ L and ⅓ L.

11. The intramedullary nail of claim 1, wherein the proximal end portion includes at least one locking hole extending through the nail body transverse to the central longitudinal axis.

12. The intramedullary nail of claim 1, wherein the distal end portion includes at least one locking hole extending through the nail body transverse to the central longitudinal axis.

13. The intramedullary nail of claim 1, wherein the radius of curvature R of the curved section is in the range of about 350 mm to about 1200 mm.

14. The intramedullary nail of claim 13, wherein the radius of curvature R of the curved section is in the range of about 400 mm to about 1100 mm.

15. The intramedullary nail of claim 1, wherein the ratio L/R is in the range of about 0.3 to about 0.7.

16. The intramedullary nail of claim 15, wherein the ratio L/R is in the range of about 0.4 to about 0.6.

17. The intramedullary nail of claim 12, wherein the distal end portion includes at least two locking holes extending through the nail body transverse to the central longitudinal axis.

18. The intramedullary nail of claim 17, wherein each of the at least two locking holes has a transverse hole axis, and the two transverse hole axes include an angle between them in the range of about 45° to about 90°.

19. The intramedullary nail of claim 17, wherein the distal end portion includes at least three locking holes extending transverse to the central longitudinal axis, the three locking holes unevenly spaced along the longitudinal central axis.

20. An intramedullary nail for use in repairing bone fractures comprising:
an elongate nail body having
a central longitudinal axis,
a proximal end portion,
a distal end portion configured and dimensioned for insertion into the medullary canal of the tibia;
a total length L, where L is in the range of about 200 mm to about 500 mm; and
a curved section having a length G, where G≦L,
wherein the curved section is defined by first and second end points, and tangents taken at the first and second end points of the curved section include an angle α in the range of about 7° to about 12° and the curved section has a radius of curvature R in the range of about 300 mm to about 1300 mm.

21. The intramedullary rod of claim 20, wherein the distal end portion is a substantially straight section having a length 1, where l≦L.

22. The intramedullary nail of claim 21, wherein the length l of the substantially straight distal end portion is in the range of about 0.20 L to about 0.55 L.
